(19) United States
(12) Patent Application Publication
(10) Pub. No.: US 2023/0302277 A1
(43) Pub. Date: Sep. 28, 2023

(54) METHOD AND SYSTEM FOR FACILITATING HEALING OR INHIBITING DEVELOPMENT OF A PULMONARY OR THROMBOSIS AILMENT

(71) Applicant: Eduard MAIBERG, Jerusalem (IL)

(72) Inventor: Eduard MAIBERG, Jerusalem (IL)

(21) Appl. No.: 18/020,012

(22) PCT Filed: Aug. 9, 2021
(86) PCT No.: PCT/IL2021/050971

§ 371 Date: Feb. 6, 2023

Related U.S. Application Data
(60) Provisional application No. 63/190,818, filed on May 20, 2021.

(30) Foreign Application Priority Data
Aug. 13, 2020 (IL) 276707

(51) Int. Cl. A61N 1/36; A61B 17/22; A61N 5/067

(52) U.S. Cl. CPC A61N 1/3601; A61B 17/22004; A61N 1/36034; A61N 5/067; A61N 2005/0659

(57) ABSTRACT

Method and system for facilitating healing or inhibiting development of a pulmonary and thrombosis ailment, such as a pulmonary lesion, a mediastinum condition of any etiology and pathogenesis, a pulmonary embolism, and a thrombotic or throm-boembolic condition. An ultrasound apparatus transmits ultrasound waves to a treatment region directed to a lung or thrombosis area od a patient, and an electrical stimulation apparatus applies electrical stimulation to the treatment region simultaneously with the ultra-sound waves transmission. Laser energy may further be applied to the treatment region. A medicant, such as systemic or transdermal thrombolytics, may also be applied. The transmitted ultrasound may be at 0.5-3 MHz frequency and at 0.5-2 W/cm2 intensity. The electrical stimulation may include electrodes of any quantity or position, such as four electrodes applying interferential stimulation. Electrical stimulation operating parameters, such as intensity, frequency, pulse duration, may vary over a treatment session, in response to clinical feedback.

### FIELD OF THE INVENTION

The present invention relates to treatment of pulmonary lesions and mediastinum conditions (“lung conditions”) of any etiology and pathogenesis, and pulmonary embolisms and other thrombotic and thromboembolic conditions.

### BACKGROUND OF THE INVENTION

As described in a recently released report of the Forum of International Respiratory Societies, four of the leading causes of death worldwide are: chronic obstructive pulmonary disease, acute respiratory tract infections, lung cancer, and tuberculosis. A fifth condition is asthma, which causes enormous global morbidity. Acute respiratory distress syndrome (ARDS) is a rapidly progressive and potentially life-threating respiratory disease resulting in dangerously low oxygen levels in the blood (hypoxemia), and is usually caused by or is a complication of a serious existing health condition, such as sepsis, pneumonia, or coronavirus disease 2019 (COVID-19). Approximately 30 percent of hospitalized COVID-19 patients develop progressive pulmonary disease. The major cause of COVID-19 mortality is respiratory failure secondary to ARDS and thrombosis. ARDS is characterized by leakage of fibrin-rich fluid from pulmonary capillaries into alveoli. It may be caused by direct binding of SARS-CoV-2 to ACE2 receptors, which regulate the production of angiotensin, on endothelial cells. Impairment of ACE2 activity may lead to activation of the kallikrein-bradykinin pathway, which in turn increases vascular permeability. Infected endothelial cells also express leukocyte adhesion molecules that recruit activated neutrophils and lymphocytes to the site of injury. The accumulation of cytokines result in a “cytokine storm” including IL-6, IL-1, IL-2, IL-10, TNF-α and IFN-γ. However, a crucial role seems to be played by IL-6, whose increased levels in the serum have been correlated with respiratory failure. Neutrophils and lymphocytes cause inflammation, loosen endothelial cell junctions, increase vascular permeability, promote alveolar fluid retention, and enhance pulmonary tissue damage.

As of now there is no effective treatment to prevent the development of these underlying problems from leading to ARDS or similar conditions, as well as for treating ARDS itself.

The current treatment protocols for these severe complications are supportive in nature, such as mechanical ventilation and prone positioning, but lack any specific treatment to the underlying local problem.

Unfortunately, the current state of the art treatments for pulmonary embolisms and other thromboembolic complications are invasive in most cases, and result in a high mortality rate.

### SUMMARY OF THE INVENTION

In accordance with one aspect of the present invention, there is thus provided a method for facilitating healing or inhibiting development of a pulmonary and thrombosis ailment, the method including the procedures of transmitting ultrasound waves to a treatment region directed to a lung or thrombosis area of a patient, and applying electrical stimulation to the treatment region simultaneously with the transmission of the ultrasound waves. The method may further include the procedures of applying laser energy to the treatment region and/or applying at least one medicant to the treatment region. The medicant may include systemic or transdermal thrombolytics for treatment of a pulmonary embolism or a thrombosis area of at least one blood vessel in at least one internal organ or limb. The ultrasound may be operative in a frequency range of 0.5 MHz to 3 MHz, and/or in an intensity range of 0.5 W/cm² to 2 W/cm². The electrical stimulation may be applied with a pulse amplitude between 0.1 mA-150 mA; a pulse duration between 1 μs-1000 μs; a frequency between 1 Hz-5000 Hz; a carrier frequency between 2000 Hz-10000 Hz; an interferential beat frequency between 1 Hz-250 Hz; between 1-2 channels; a constant current (CC) mode or constant voltage (CV) mode; a burst frequency between 0-75; a sweep low beat frequency between 1-199; and/or a sweep high frequency between 2-200. The applied laser energy may be applied at a wavelength of approximately 830 nm, with an energy density of approximately 9 J/cm², with a power of approximately 35 mW, and/or with a duration of approximately 80 seconds per treatment point. The pulmonary and thrombosis ailment may include: a pulmonary lesion; a mediastinum condition of any etiology and pathogenesis; a pulmonary embolism; and/or a thrombotic or thromboembolic condition.

In accordance with another aspect of the present invention, there is provided a system for facilitating healing or inhibiting development of a pulmonary and thrombosis ailment, the system including an ultrasound apparatus configured to transmit ultrasound waves to a treatment region directed to a lung or thrombosis area of a patient, and an electrical stimulation apparatus, configured to apply electrical stimulation to the treatment region simultaneously with the transmission of the ultrasound waves. The system may further include a laser apparatus, configured to apply laser energy to the treatment region. At least one medicant may be applied to the treatment region. The medicant may include systemic or transdermal thrombolytics for treatment of a pulmonary embolism or a thrombosis area of at least one blood vessel in at least one internal organ or limb. The ultrasound may be operative in a frequency range of 0.5 MHz to 3 MHz, and/or in an intensity range of 0.5 W/cm² to 2 W/cm². The electrical stimulation may be applied with a pulse amplitude between 0.1 mA-150 mA; a pulse duration between 1 μs-1000 μs; a frequency between 1 Hz-5000 Hz; a carrier frequency between 2000 Hz-10000 Hz; an interferential beat frequency between 1 Hz-250 Hz; between 1-2 channels; a constant current (CC) mode or constant voltage (CV) mode; a burst frequency between 0-75; a sweep low beat frequency between 1-199; and/or a sweep high frequency between 2-200. The applied laser energy may be applied at a wavelength of approximately 830 nm, with an energy density of approximately 9 J/cm², with a power of approximately 35 mW, and/or with a duration of approximately 80 seconds per treatment point. The pulmonary and thrombosis ailment may include: a pulmonary lesion; a mediastinum condition of any etiology and pathogenesis; a pulmonary embolism; and/or a thrombotic or thromboembolic condition.

### DETAILED DESCRIPTION OF THE EMBODIMENTS

The present invention overcomes the disadvantages of the prior art by providing methods and systems for the treatment of pulmonary lesions and mediastinum conditions (“lung conditions”) of any etiology and pathogenesis, as well as pulmonary embolisms and other thrombotic and thromboembolic conditions, collectively referred to herein as “pulmonary and thrombosis ailments”, by a new form of energy which is a result of a mix and combination of electric field and ultrasound energy. This combination can be used alone or with therapeutic laser, with or without systemic or locally applied medicines. For example, the disclosed treatment may be used for treating patients with acute or subacute pulmonary pathologies, such as lung lesions caused by coronavirus disease 2019 (COVID-19) or other ailments that may progress into acute respiratory distress syndrome (ARDS) or other forms of respiratory failure. The treatment may be applied as a prophylactic or as a healing measure.

The term “pulmonary and thrombosis ailment”, and variations thereof, is used herein to broadly refer to any form of acute or subacute pulmonary pathology or ailment which can cause alveolar and/or vascular damage, including pulmonary embolisms and other thrombotic and thromboembolic conditions, such as a thrombosis area of one or more blood vessels in one or more internal organs or limbs. An example of a pulmonary and thrombosis ailment may include pulmonary lesions manifested during ARDS caused by COVID-19. It is appreciated that ARDS caused by COVID-19 generally has very similar pathophysiological mechanisms to other pulmonary conditions characterized by massive alveolar and vascular damages, and therefore may serve as a useful example for the disclosed treatment.

### Discovery History

The first and fundamental difference of our approach is that after we carefully studied the histopathological changes in the lungs in patients with ARDS of various etiologies, we have identified criteria that allows us to refer to what is happening in the lungs a “vascular wound with a poor prognosis for healing”. We assume that before us no one used such a formulation in relation to lung lesions, nevertheless, it clearly reflects the essence of what is happening and gave us the key to choosing the right treatment method.

Following this approach, we searched for similar poor healing prognosis wounds in other parts of the human body, in the hope that if enough similarities are found we can then adopt the accepted treatments from those wounds to the aforesaid “pulmonary and thrombosis ailments”. For comparison we chose the following wounds which have a poor healing prognosis: diabetic foot ulcers, venous leg ulcers and pressure ulcers.

While analyzing these wounds we found extremely similar levels of the main indicators, namely: IL6, MMP-9, TNF-α, D-dimer, TGF-beta1, which, without connection to the wound etiology, determine the ultimate severity in the healing of lesions.
1. Diabetic leg: studies demonstrate that diabetic subjects with various grades of diabetic foot ulcer showed significantly higher IL-6, in comparison with diabetes without foot ulcer, independent of concomitant infections.
2. TNF-α also showed significantly higher values in comparison with diabetes without foot ulcers.
3. TNF-α has been shown to be present in significantly higher levels in chronic nonhealing venous ulcers than in acute healing wounds.
4. MMP-9 levels have a significant deleterious effect on diabetic foot ulcer healing. The pattern of increased MMP-9 in poorly healing ulcers was observed in various types of diabetic foot ulcers, suggesting that it is more strongly linked with the healing process rather than with an underlying etiology. Elevation of MMP-9 in chronic wound fluid correlates with a clinically worse wound.
5. Significant changes in D-dimer levels indicate diabetes disease progression to macrovascular complications. Post-thrombotic syndrome (PTS) is the most frequent chronic complication of acute deep vein thrombosis (DVT), occurring in 20-40% of patients with DVT, strongly associated with chronic vascular ulcer and pulmonary embolism (PE).
6. Human Pressure Ulcers: significantly increased level of IL-6 that is a bad prognostic factor and levels of MMP-9 also elevated more than 25-fold in fluids from pressure ulcer compared with fluids from healing wounds.
7. The lack of TGF-beta1 up-regulation in both diabetic foot ulcers and venous ulcers may explain the impaired healing in these chronic wounds, and could represent a general pattern for chronicity.

In turn, a clear correlation was established between same, above mentioned cytokines and poor prognosis of lung injuries:
1. High serum levels of IL-6, plays a critical role in the severity of the disease and is almost always associated with poor prognosis and lung lesions in the acute and later stages
2. TNF-α was significantly higher in critically ill patients than in non-critically ill patients
3. Matrix metalloproteinases MMP-9. In acute lung injury, MMP-9 released from neutrophils promotes inflammation and degradation of the alveolar capillary barrier, further stimulating migration of inflammatory cells, and is a strong indicator of respiratory failure
4. Patients with higher levels of D-dimer and those requiring intubation were at a higher risk of developing a pulmonary embolism (PE)
5. TGF-β. Sudden and uncontrolled increases in active (possibly with the help of some proinflammatory cytokines such as TNFα, IL-6, and IL-1β TGF-β, inevitably result in rapid and massive edema and fibrosis that remodels and ultimately blocks the airways. This leads to the functional failure of the lungs and death of the patients.

Thus, it is difficult not to assume that we are talking about rather similative processes, despite the completely different etiology. Both of them are united by wounds existing at advanced stages, which are characterized by very poor healing, including those associated with the predominant vascular component with coagulation problems and similar expression of the aforementioned proinflammatory cytokines that are responsible for poor wound healing and poor prognosis in both cases.

Unfortunately, the treatment of poorly healing vascular wounds of any localization has remained a serious problem associated with high morbidity and mortality. Nevertheless, over the past few years significant success has been reported with combination of electric field with treatment ranges of ultrasound in the healing of diabetic foot ulcers, venous leg ulcers and pressure sores. 70-75% of the wounds show a closure rate of at least 50%. The results of numerous studies certainly support the statement that this combination has an immediate effect (after 1-2 treatments) on wounds that had been stagnant for a minimum of 30 days.

Thus, summarizing all of the above, we concluded that the use of the aforementioned new energy created as a result of combination of therapeutic ultrasound with electric field has great potential for the treatment of poorly healing vascular wounds, both due to its thrombolytic and anti-inflammatory ability and the ability to regenerate and accelerate all stages of regeneration of a wide variety of tissues, including pulmonary, vascular, nervous, etc. This combination of two energies may be an extremely effective and non-invasive method for treating pulmonary and thrombosis ailments, regardless of their etiology, nature and severity.

### Mechanism of Action and Conclusions

Numerous studies over decades have shown the unique properties of therapeutic ultrasound, including anti-inflammatory, regenerative, and thrombolytic. Nevertheless, there is absolutely a discrepancy between the above properties and the minimal clinical effect when external ultrasound is applied. At the same time, introduction of an ultrasound (US) transducer directly into the pulmonary artery in the treatment of a pulmonary embolism (PE) may have a pronounced effect.

Thus, a main problem is to maintain the therapeutic effect of ultrasound as it passes through various organs and tissues that have different impedances. The key to solving this problem is the creation of the new energy consisting of a combination of ultrasonic and electric field energies. The electric field makes it possible to equalize the impedance of different type of tissues, which makes it possible for the ultrasonic wave to exert its effect unhindered and uniformly. Currently, the use of electric field and ultrasonic waves in therapeutic ranges in acute conditions of the lungs and mediastinum, is mostly contraindicated. However, we have not found any evidence-based work to substantiate this.

Moreover, given the use of an ultrasound probe that is directly inserted into the pulmonary artery for the treatment of PE with absence of side effects because of US, it is the best evidence of the safety of this treatment method in the lung area that was historically considered a contraindication for ultrasound therapy.

### Conclusions

1. The use of external ultrasound alone in therapeutic modes in the lung area and mediastinum has a thrombolytic effect and thus can be effective for restoring the work of the alveolar vascular complex. The above effect occurs as a consequence of the phenomenon of cavitation, which is traditionally considered a side effect of ultrasound treatment, however, we established that it is actually the cavitation in the therapeutic ranges of ultrasound that can destroy multiple blood clots without damaging the lung tissue. Obviously, diagnostic ranges of US cannot have any therapeutic effect, while in combination with an electric field it can have a moderate therapeutic effect on damaged lung tissue.

Nevertheless, the thrombolytic effect and other effects of therapeutic ultrasound described in the literature, have minimal clinically importance, especially in the chest area. The reason of this is that even when using treatment modes of US, we can talk about a very small penetration (5-8 cm) of waves in this area and the main issue is that after several minutes of treatment the tissues change their impedance which brings to a halt the penetration of ultrasound waves.
2. In contrast, the combination of therapeutic ultrasound and an electric field in patients with pulmonary and thrombosis ailments, especially acute ones, will be very effective. The electric field manages the bio-impedance which keeps the gateways open, allowing the full ultrasonic energy to reach the affected tissue over the entire duration of a treatment session. This will make it possible to carry out a full-fledged local treatment of pulmonary ailments of various etiologies and severities. We expect to obtain a very rapid effect of regeneration of the alveolar vascular complex and restoration of the lung structure, which should lead to normal lung function and solve thrombotic and inflammatory complications
3. As stated above, the combination of ultrasound with electric field allows the new energy to reach the required localization in full. We expect a quick effect when using therapeutic ultrasound together with an electric field and thrombolytics administered either systemically (e.g., streptokinase, urokinase, etc.) or transdermally (e.g., phonophoresis) in the treatment of a pulmonary embolism. In addition, this new energy can be successfully used in the treatment of thrombosis areas of small and large vessels of various localizations, including the heart and brain, as well as internal organs and limbs. Currently, according to FDA approved technology an ultrasound probe is surgically inserted directly into the pulmonary artery and together with thrombolytic agents helps with pulmonary embolism (PE) in 85% of the cases. We believe that the proposed noninvasive therapy with possibly adding systemic administrated or locally applied agents can be a successful alternative for treatment of PE, but without the operational risks and other complications associated with invasive treatment of blood clots.
4. We assume that with the combination of two energies such as electric fields and ultrasound, due to local micro-massage capability that these energies possess, it will be possible to mobilize an additional 10-15% of the non-active areas of the lung. Thus, perfusion in these areas can be significantly improved.

### Technical Parameters

The operational parameters of the ultrasound, the electric field, and the laser may be selected to ensure both safety and effectivity. For example, the frequency range of the applied therapeutic ultrasound is in the range of 0.5 MHz to 3.0 MHz (and in some cases can reach up to 20 MHz). An exemplary operational intensity is in the range of 0.5 W/cm² to 2.0 W/cm².

It is noted that intensities and frequencies of diagnostic ultrasound (i.e., applied solely and for diagnostic purposes) are generally not effective for therapeutic treatment in accordance with the present invention, but can play some role in combination with electric field as described below.

Exemplary operational parameters for the applied laser specifications may include the following: 830 nm wavelength, 9 J/cm² energy density, 35 mW power, 80 seconds duration per point, and 3 points per application.

The terms “electric field” and “electrical stimulation”, and grammatical variations thereof, are used interchangeably herein to refer to the application of electrical fields or electromagnetic fields, or electrical/electromagnetic energy, to stimulate a treatment region, such as via one or more electrodes.

According to an embodiment of the present invention, electrical stimulation is applied toward the treatment region simultaneously with the ultrasound application. The electrical stimulation may be applied using any number of electrodes (e.g., 1, 2 or 4 electrodes), which may be positioned at a suitable location on the patient body and directed to the treatment region. The electrodes may be adhered or otherwise affixed onto the patient skin or implanted, so that they remain stationary during the treatment process. The electrodes may alternatively be integrated with the ultrasound transducer such that the electrodes are moved and operated in conjunction with the ultrasound transducer.

As mentioned above, the electric field combined with ultrasonic energy allows to create a new energy with a cumulative effect and, accordingly, achieve the desired clinical result from the new energy.

For electrical stimulation, a wide variety of methods may be used, including but not limited to:
1. TENS (Transcutaneous Electrical Nerve Stimulation) with all its modifications.
2. Interferential Therapy.
3. EMS (Electrical Muscle Stimulation) with an intensity of more than 50 mA.
4. Direct current (DC) is a sinusoidal wave.
5. Russian current as an example of burst-modulated AC (BMAC). Recently developed devices have been designed to deliver different configurations of the traditional Russian current with adjustable levels of carrier frequency (e.g., 1000-5000 Hz), burst frequency (e.g., 50-75 bursts per second), or burst duration (e.g., 2-10 ms).
6. Pulsed current (PC). With PC, the duration of the pulse is very short, typically only a few hundred microseconds (one-millionth of a second), and the total charge delivered using PC is extremely low.
7. Monophasic pulsed current flows only in one direction, and the polarity of the electrodes does not change. Most often, monophasic pulsed current appears as a rectangular waveform, with a range of pulse amplitude, pulse duration (commonly 100-400 μs), and pulse frequency (commonly 50-100 Hz).
8. High-voltage pulsed current (HVPC), biphasic symmetrical pulsed current, biphasic asymmetrical pulsed current.
9. Galvanic Stimulation.
10. Percutaneous Electrical Nerve Stimulation.
11. Pulsed Electromagnetic Field Therapy.

Waveforms can be used as monophasic or biphasic, described by their shape (e.g., monophasic rectangular, symmetrical biphasic rectangular, asymmetrical biphasic rectangular, sinusoidal).

All of the above listed types of electrical stimulation, as well as similar ones, may have a small independent effect for the treatment of pulmonary and thrombosis ailments and are mostly contraindicated for them. However, in combination with ultrasound energy and when choosing their correct modes, they may have a significant clinical effect. The type of electrical stimulation combined with ultrasound waves is determined based on the localization, massiveness, clinical severity and imaging of the clinical condition of the patient.

For example, when treating a patient with a small lesion in the apex of the lung, then can apply a low frequency (typically between 0-300 Hz) of pulsed current, i.e. a current in which the unidirectional or bidirectional flow of current periodically ceases over time. The pulse durations may be between about 1 μs and 1000 μs with asymmetrical biphasic rectangular or symmetrical biphasic rectangular waveforms. The pulse amplitude is generally low (e.g., <50 mA).

For more intense lesions, can use a modified square direct current with monophasic pulses changing polarity at regular intervals (e.g., 0.4 s) and delivered by two electrodes, pulse amplitude is low (e.g., 1-600 μA) with no paresthesia frequency (e.g., 1-5000 Hz.) High frequency, low intensity, continuous pattern, or low frequency high intensity burst pattern, or high frequency/high intensity continuous pattern can be used according to the specific situation.

In cases where most of the lung is affected, interferential electrical stimulation can be used. Two out-of-phase currents which interfere with each other to produce an amplitude-modulated wave traditionally delivered by four electrodes. The pulse amplitude is low (e.g., up to 50 mA), the amplitude-modulated frequency is approximately 1-200 Hz, and the carrier wave frequencies are approximately 2.

In case of localization of the lesions in the depth of the lungs, then Russian current as an example of burst-modulated AC (BMAC) can be used. This classic waveform is a medium-frequency sinusoidal current that is balanced and switches polarity 2,500 times per second (2500 Hz). A type of BMAC, Russian current is interrupted (modulated) into 20-millisecond bursts, consisting of 10 milliseconds of AC current followed by 10 milliseconds of no AC current (50% duty cycle). This is repeated 50 times per second (burst rate of 50). The background 2500-Hz AC is called the carrier frequency.

The ultimate choice of electrode position and quantity, depends upon an accurate assessment of the cause and location of the lesion in the lungs and also the type of electrotherapy which is to be used.

In an exemplary operational session, 4 electrodes located diagonally were used with interferential electrical stimulation, however other types of electrical stimulation may also be applied. The duration of a session may vary from 10 minutes to 1 hour, depending on the complexity of the patient's condition. The electrical stimulation may be used as part of electropuncture, in wireless mode and using a mobile electrode. Operating parameters of the electrical stimulation, such as the intensity, frequency, and/or pulse duration, may vary over the course of a treatment session, such as in response to clinical feedback (e.g. pain or discomfort).

A system for facilitating treatment of patients with a pulmonary and thrombosis ailment in accordance with an embodiment of the present invention may include at least: an ultrasound apparatus, an optional laser apparatus, an electrical stimulation apparatus, and a controller. The ultrasound apparatus is configured to generate and apply ultrasound energy, and may include a signal generator unit and at least one ultrasound transducer. The laser apparatus is configured to generate and apply laser radiation, and may include a laser energy generator, and a laser applicator such as a handheld laser probe. The electrical stimulation apparatus is configured to generate and apply electrical stimulation, and may include one or more electrodes. The controller is configured to control and manage the operation of the ultrasound apparatus, the electrical stimulation apparatus, and/or the laser apparatus. The controller may be partially or fully embodied by any form of hardware, software, or a combination thereof, and may be at least partially embodied by a hardware or software component integrated with at least one component of the ultrasound apparatus, electrical stimulation apparatus, and/or the laser apparatus. The functionality associated with each of the system elements may be distributed among multiple devices or components (e.g., such as a dedicated controller for each one of the ultrasound apparatus and the laser apparatus).

In an example treatment session, 5 patients suffering from ARDS were treated. All of them before treatment were in an extremely severe condition and coma, connected to mechanical ventilation machines, and one patient to an extracorporeal membrane oxygenation (ECMO) machine.

The results clearly show that after 7-10 days of treatment, there was significant clinical and x-ray improvement with the restoration of the pulmonary structure. Currently, permission has been obtained to conduct a clinical trial for 40 people, where the second lung will be the control.

Thus, a therapeutic treatment in accordance with the inventions of the present invention may include:
1. US in therapeutic ranges combined simultaneously with electric field for treatment of acute or chronic “lung conditions”.
2. US in therapeutic ranges combined simultaneously with electric field for treatment of pulmonary embolism.
3. US in therapeutic ranges combined simultaneously with electric field for treatment of thrombosis area of small and large vessels of various localization, including the heart and brain, as well as internal organs and limbs.
4. Items 1-3 above with systemic or transdermal thrombolytic medication.
5. Items 1-3 above with systemic or locally applied anti-inflammatory, scar lises (etc.) medications.
6. Items 1-5 above where the US ranges are operative in a frequency range of 0.5 MHz to 3 MHz and an intensity range of 0.5 W/cm² to 2 W/cm².
7. Items 1-5 above where the US ranges are operative in a frequency range of 0.5 MHz to 3 MHz and an intensity range of 0.5 W/cm² to 2 W/cm². The electric field ranges are: The pulse amplitude used in this method is between 0.1 mA-150 mA. The pulse duration is between 1 μs-1000 μs (might reach 0.5 seconds). The frequency is between 1-5000 Hz. The carrier frequency is between 2000-10000 Hz, the beat frequency is between 1-250 Hz (for interferential). The number of channels is between 1-2 channels. The modes used are: constant current (CC) or constant voltage (CV), a burst frequency between 0-75, a sweep low beat frequency between 1-199 and a sweep high frequency between 2-200.

While certain embodiments of the disclosed subject matter have been described, so as to enable one of skill in the art to practice the present invention, the preceding description is intended to be exemplary only. It should not be used to limit the scope of the disclosed subject matter, which should be determined by reference to the following claims.

1. A method for facilitating healing or inhibiting development of a pulmonary and thrombosis ailment, the method comprising the procedures of:
transmitting ultrasound waves to a treatment region directed to a lung or thrombosis area of a patient; and
applying electrical stimulation to said treatment region simultaneously with the transmission of the ultrasound waves.

2. The method of claim 1, further comprising the procedure of applying laser energy to said treatment region.

3. The method of claim 1, further comprising the procedure of applying at least one medicant to said treatment region.

4. The method of claim 3, wherein the medicant comprises systemic or transdermal thrombolytics for treatment of a pulmonary embolism or a thrombosis area of at least one blood vessel in at least one internal organ or limb.

5. The method of claim 1, wherein the ultrasound is operative in a frequency range of 0.5 MHz to 3 MHz.

6. The method of claim 1, wherein the ultrasound is operative in an intensity range of 0.5 W/cm² to 2 W/cm².

7. The method of claim 1, where the electrical stimulation comprises at least one parameter selected from the group consisting of:
a pulse amplitude between 0.1 mA-150 mA;
a pulse duration between 1 μs-1000 μs;
a frequency between 1 Hz-5000 Hz;
a carrier frequency between 2000 Hz-10000 Hz;
an interferential beat frequency between 1 Hz-250 Hz;
between 1-2 channels;
a constant current (CC) mode or constant voltage (CV) mode;
a burst frequency between 0-75;
a sweep low beat frequency between 1-199; and
a sweep high frequency between 2-200.

8. The method of claim 2, wherein the applied laser energy comprises at least one parameter selected from the group consisting of:
a wavelength of approximately 830 nm;
an energy density of approximately 9 J/cm²;
a power of approximately 35 mW; and
a duration of approximately 80 seconds per treatment point.

9. The method of claim 1, wherein the pulmonary and thrombosis ailment is selected from the group consisting of:
a pulmonary lesion;
a mediastinum condition of any etiology and pathogenesis;
a pulmonary embolism;
a thrombotic or thromboembolic condition.

10. A system for facilitating healing or inhibiting development of a pulmonary and thrombosis ailment, the system comprising:
an ultrasound apparatus, configured to transmit ultrasound waves to a treatment region directed to a lung or thrombosis area of a patient; and
an electrical stimulation apparatus, configured to apply electrical stimulation to said treatment region simultaneously with the transmission of the ultrasound waves.

11. The system of claim 10, further comprising a laser apparatus, configured to apply laser energy to said treatment region.

12. The system of claim 10, wherein at least one medicant is applied to said treatment region.

13. The system of claim 12, wherein the medicant comprises systemic or transdermal thrombolytics for treatment of a pulmonary embolism or a thrombosis area of at least one blood vessel in at least one internal organ or limb.

14. The system of claim 10, wherein the ultrasound is operative in a frequency range of 0.5 MHz to 3 MHz.

15. The system of claim 10, wherein the ultrasound is operative in an intensity range of 0.5 W/cm² to 2 W/cm².

16. The system of claim 10, where the electrical stimulation comprises at least one parameter selected from the group consisting of:
a pulse amplitude between 0.1 mA-150 mA;
a pulse duration between 1 μs-1000 μs;
a frequency between 1 Hz-5000 Hz;
a carrier frequency between 2000 Hz-10000 Hz;
an interferential beat frequency between 1 Hz-250 Hz;
between 1-2 channels;
a constant current (CC) mode or constant voltage (CV) mode;
a burst frequency between 0-75;
a sweep low beat frequency between 1-199; and
a sweep high frequency between 2-200.

17. The system of claim 11, wherein the applied laser comprises at least one parameter selected from the group consisting of:
a wavelength of approximately 830 nm;
an energy density of approximately 9 J/cm²;
a power of approximately 35 mW; and
a duration of approximately 80 seconds per treatment point.

18. The system of claim 10, wherein the pulmonary and thrombosis ailment is selected from the group consisting of:
a pulmonary lesion;
a mediastinum condition of any etiology and pathogenesis;
a pulmonary embolism;
a thrombotic or thromboembolic condition.
